(19) United States
(12) Patent Application Publication
(10) Pub. No.: US 2013/0085556 A1
(43) Pub. Date: Apr. 4, 2013

(54) THERMAL BODY-CARE ELEMENT AND METHOD OF USE OF SAME

(71) Applicants: Ronald J. Gillespie, North Brunswick, NJ (US); Robert Hamlen, Holmdel, NJ (US); Raymond J. Hull, JR., Hampton, NJ (US); Michael H. Kuchavik, SR., Bath, PA (US)

(72) Inventors: Ronald J. Gillespie, North Brunswick, NJ (US); Robert Hamlen, Holmdel, NJ (US); Raymond J. Hull, JR., Hampton, NJ (US); Michael H. Kuchavik, SR., Bath, PA (US)

(21) Appl. No.: 13/249,386

(22) Filed: Sep. 30, 2011

(51) Int. Cl. A61F 7/08

(52) U.S. Cl. 607/114

(57) ABSTRACT

A thermal body-care element includes a container having at least one thermally conductive surface enclosing an electric heater in thermal contact with a thermal energy storage medium comprising a phase change storage medium and an electrical connection for selective coupling to an external electrical power source. The external electrical power source is external to the thermal body-care element. A method of applying heat to a skin surface of a human or animal is also disclosed.

### BACKGROUND OF THE INVENTION

The present invention relates to a system for applying heat to the skin of a body, such as a human body. The heat application surface is small, lightweight, and cordless.

Heated, skin care devices are known. Some of these provide motion, such as heated massagers, while others simply apply heat for therapeutic, cosmetic, and/or other purposes.

Burkardt, U.S. Pat. No. 2,985,166, purports to disclose a heated massaging device including both a motor to provide vibrating motion to the skin and a heating coil to provide heat to the skin. However, in order to provide the power for both motion and heat, the device is directly connected to an external power source, such as a household electrical receptacle. The device is not cordless.

Rhoades, US Pub. Pat. App. No. 2003/0165550 A1, purports to disclose a battery-operated vibrating microdermabrasion device that may include a heating unit disposed within or adjacent to the device. While the embodiments described in detail in Rhoades are cordless, there is no detail how the heating unit would be included, and it is not clear that such a unit could be cordless in operation.

Gebhard, U.S. Pat. No. 6,001,070, purports to disclose a cordless facial iron that incorporates rechargeable batteries. The facial iron has a spoon shaped heating surface for applying heat to a user's skin. The heating surface is powered by the rechargeable batteries and is activated by a thermostatically controlled circuit. This cordless device lacks a motor to provide motion to the heating surface that contacts the user's skin.

Finally, Li et al., U.S. Pat. No. 6,245,093, purports to disclose several embodiments of an apparatus to treat skin itch and skin rash. The apparatus includes a body heater that can apply heat with a cycle time and pulse. In one embodiment, a motor and crank system moves the heating unit into and away from skin contact to provide this cyclic heating. However, such a movement of the heating unit does not provide substantial movement of the skin that is provided in motions such as vibration, reciprocation, oscillation, rotation, and the like.

Despite the teaching of the prior art, there is a continuing need for skin care devices that provide sufficient heat for a long enough time to deliver the benefits of a heated surface with the portability of a manual or battery-powered device.

### SUMMARY OF THE INVENTION

We have discovered that it is possible to provide the desired portability by de-coupling the power to heat the device from the power (if any) to provide motion to the skin-contacting surface of the device.

In one embodiment, a thermal body-care element includes a container having at least one thermally conductive surface enclosing an electric heater in thermal contact with a thermal energy storage medium comprising a phase change storage medium and an electrical connection for selective coupling to an external electrical power source. The external electrical power source is external to the thermal body-care element.

In another aspect of the invention, a method of applying heat to a skin surface of a human or animal includes a) electrically connecting an electric heater to an external electrical power source to heat a thermal energy storage medium in thermal contact therewith; b) disconnecting the electric heater from the external electrical power source; and c) applying the body-care surface to the skin surface while the electric heater is disconnected from the external electrical power source. The electric heater and thermal energy storage medium are in a container arranged and configured for gripping by a human hand.

### BRIEF DESCRIPTION OF THE DRAWING

FIG. 1 is a side elevation of a skin care device according to the present invention.

FIG. 2 is an end view of the skin care device of FIG. 1.

FIG. 3 is a cross-section of the skin care device of FIGS. 1 and 2 taken along section line 3-3 of FIG. 2.

FIG. 4 is a perspective view of a thermal body care element, useful in the device of FIGS. 1-3.

FIGS. 5A-E are various views of the thermal body care element of FIG. 4.

FIG. 5A is a bottom plan view of the heat-generating element.

FIG. 5B is a side elevation of the heat-generating element.

FIG. 5C is a cross-section of the heat-generating element taken along section line 5C-5C of FIG. 5A.

FIG. 5D is a cross-section of the heat-generating element taken along section line 5D-5D of FIG. 5A.

FIG. 5E is a cross-section of the heat-generating element taken along section line 5E-5E of FIG. 5B.

FIG. 6A is side elevation of an alternative thermal body-care element according to the present invention.

FIG. 6B is bottom plan view of the alternative thermal body-care element of FIG. 6A.

FIG. 7A is a perspective view of an energizer stand useful with the body-care device of FIG. 1.

FIG. 7B is a side elevation of the energizer stand shown in FIG. 7A with a body-care device of FIG. 1 placed thereon.

FIG. 8 is a partially exploded, perspective view of an alternative skin care device according to the present invention.

FIG. 9 is a perspective view of an attachment surface of a thermal body-care element designed to be used with the skin care device of FIG. 8.

FIG. 10 is an end view of the skin care device of FIG. 8.

FIG. 11 is a side elevation of an energizer stand with the body-care device of FIG. 8 placed thereon.

FIG. 12 is an electrical schematic of one embodiment of an energizer stand and a thermal body-care element according to the present invention.

FIG. 13 is an exploded perspective view of elements of the thermal body care element of FIGS. 4-5.

### DETAILED DESCRIPTION OF THE PREFERRED EMBODIMENTS

The challenge that has faced and continues to face developers of commercial heated, skin care devices relates to providing sufficient heat for a long enough time to deliver the benefits of a heated surface with the portability of a manual or battery-powered device. We have discovered that it is possible to provide the desired portability by de-coupling the power to heat the device from the power (if any) to provide motion to the skin-contacting surface of the device. A number of preferred embodiments of the invention are discussed in this section.

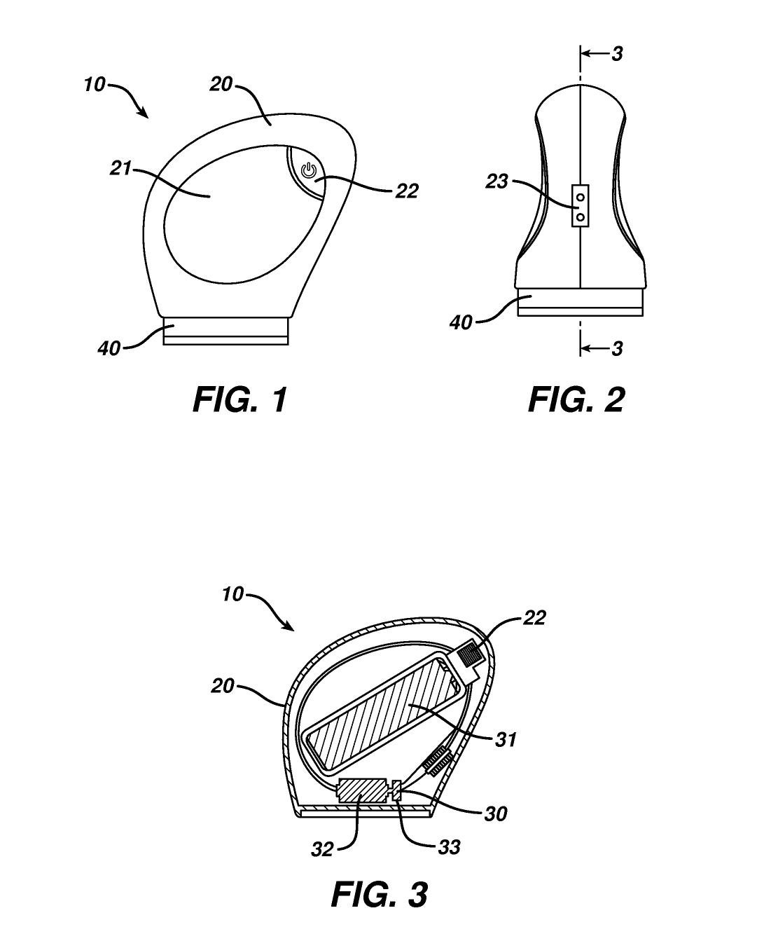

The first preferred embodiment may be understood by referring to FIGS. 1-5 and 7. In FIGS. 1-3, a cordless, handheld, skin care device 10 includes a housing 20 that contains a motion-generating system 30 with a thermal body-care element 40 attached thereto. The housing 20 includes one or more gripping surfaces 21, a power switch 22, and electrical contacts 23.

The motion-generating system 30 is shown in more detail in FIG. 3. This includes a power source, such as one or more batteries 31, coupled to a motor 32 through the power switch (element 22 shown in FIGS. 1-3). The motor 32 may be coupled to the thermal body-care element 40 in a manner to transfer vibrating motion as known to those of ordinary skill in the art. For example, vibrations may be generated by means of an eccentrically mounted weight 33 on the motor shaft. Alternatively, the motor 32 may be coupled to the thermal body care element 40 via gears or other transfer mechanisms to provide rotation, reciprocation, oscillation, or other motions to the thermal body care element 40. The transfer mechanism may include one or more clutches to permit the system to selectively transfer one or more of these motions, as desired.

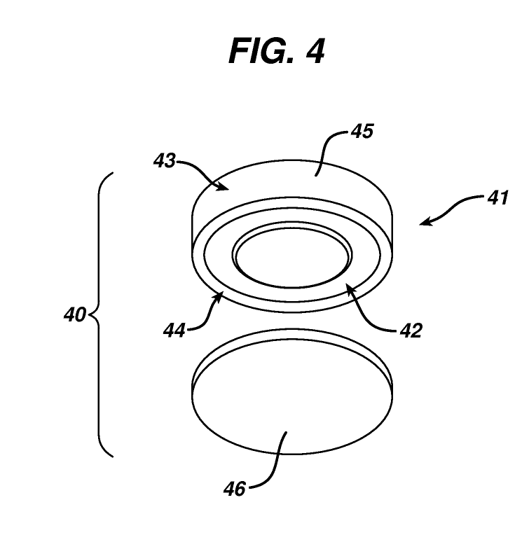

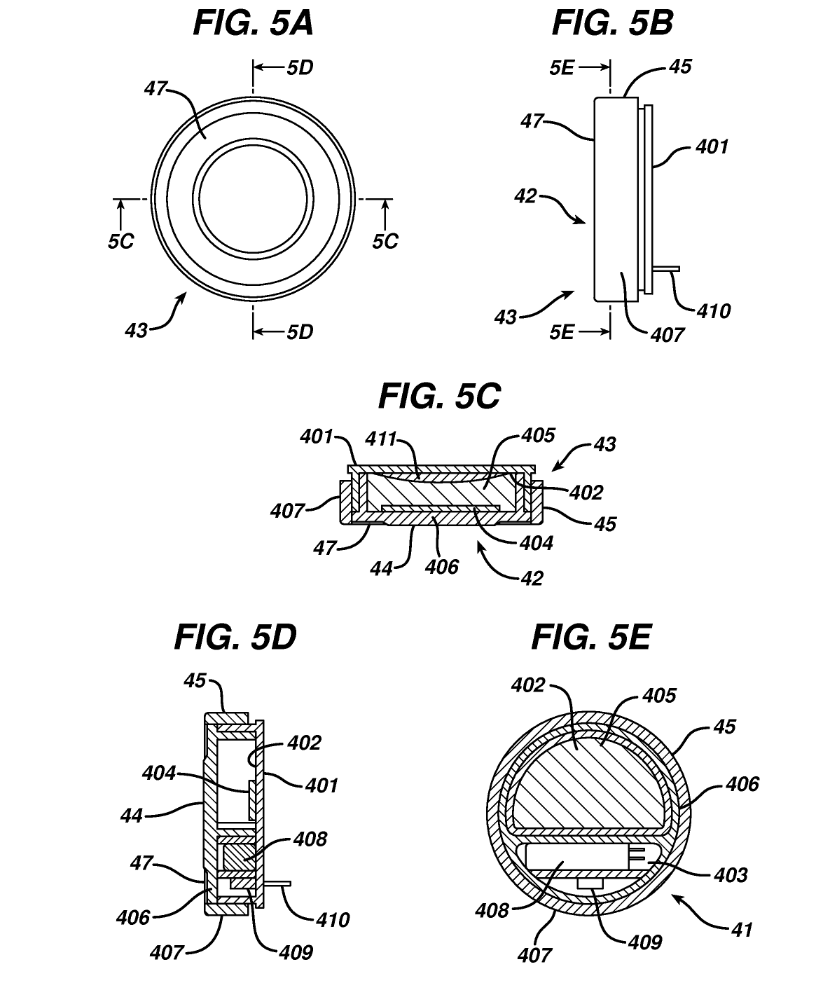

The thermal body-care element 40 is shown in more detail in FIGS. 4-5. Referring to FIG. 4, thermal body-care element 40 is a compact, self-contained heating system, and it includes a heat-generating element 41 that contains a heat source (described more fully, below) thermally coupled to a body-care surface 42. The heat-generating element 41 includes a container 43 having at least one thermally conductive surface 44 and one or more thermally insulating surfaces 45 enclosing the heat source and a thermal energy storage medium. The container 43 is sealed to contain the heat source and thermal energy storage medium to provide a safe heating system for use by consumers in their homes. A supplemental body-care component 46 may be releasably coupled to the body-care surface 42 via a coupling structure 47, which may include a plurality of plastic hooks (available from Velcro USA Inc., Manchester, N.H., USA).

In a preferred embodiment shown in detail in FIGS. 5A-5E, the container 43 of the thermal body-care element 40 includes a base 401 formed of a thermally insulating material that at least partially defines a first chamber 402 and a second chamber 403 separated by a wall 401a. The first chamber 402 contains a heat source, such as an electric heater 404 and the thermal energy storage medium 405 (indicated by cross-hatching in FIGS. 5C and 5E). The container also includes a thermally conductive cap 406 that cooperates with the base 401 to enclose the first chamber 402 and the second chamber 403. The cap 406 has walls 406a extending into the first chamber 402 to improve thermal contact with the thermal energy storage medium 405 and a flange 406b extending into the second chamber 403. The container 43 may also include additional thermal insulating surfaces 45 in the form of an external sleeve 407 to contain one or more portions of the thermally conductive cap 406. Other portions of the thermally conductive cap 406 may form the body-care surface 42 of the container 43.

The heat-generating element 41 is heated by means of electric heater 404 contained within the first chamber 402. The electric heater 404 is in thermal contact with the thermal energy storage medium 405 also contained within first chamber 402. Thus, when the electric heater 404 is energized, it heats the thermal energy storage medium 405. The electric heater 404 is preferably controlled, at least in part, by a thermal switch 408, and the heating system is preferably protected from overload by a safety cut-off switch 409. These two switches are mounted in thermal contact with the flange 406b of the cap 406 to enable them to sense the temperature of the first chamber 402, during use, and they are arranged along the electrical power supply circuit between the electric heater 404 and internal electrical contacts 410 that are connectable to an external power source, described below.

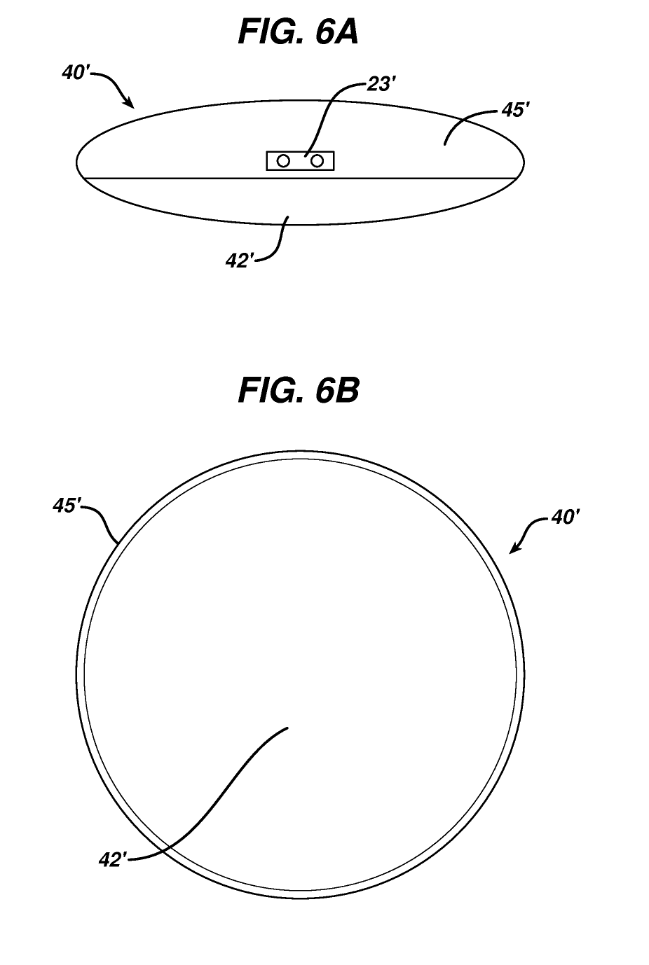

While the thermal body-care element 40 has been shown in use with the handheld housing 20 having the motion-generating system 30, it will be recognized that the thermal body-care element 40 may be used, and shaped appropriately, without the housing 20 and motion-generating system 30. The thermal body-care element 40 can keep the geometries shown in FIGS. 4 and 5A-5E, or it may take on a different form factor, such as shown in FIG. 6.

As shown in FIGS. 6A and 6B, the thermal body-care element 40′ is a hot stone replica that is capable of generating heat instead of being heated in a water bath or other heating system. Again, this thermal body-care element 40′ is an improvement over existing, natural hot stones as it is capable of maintaining a desired temperature, as described in greater detail, below. The thermal body-care element 40′ may be generally circular when viewed from the top or bottom (FIG. 6B), and elliptical when viewed from the side (FIG. 6A). The thermal body-care element 40′ again has a thermally conductive body-care surface 42′, thermally insulating surfaces 45′ and contains a heat-generating element. The thermal body-care element 40′ also has electrical contacts 23′ disposed on an outer surface of the thermal body-care element 40′ for selective coupling the heat-generating element to an external electrical power source.

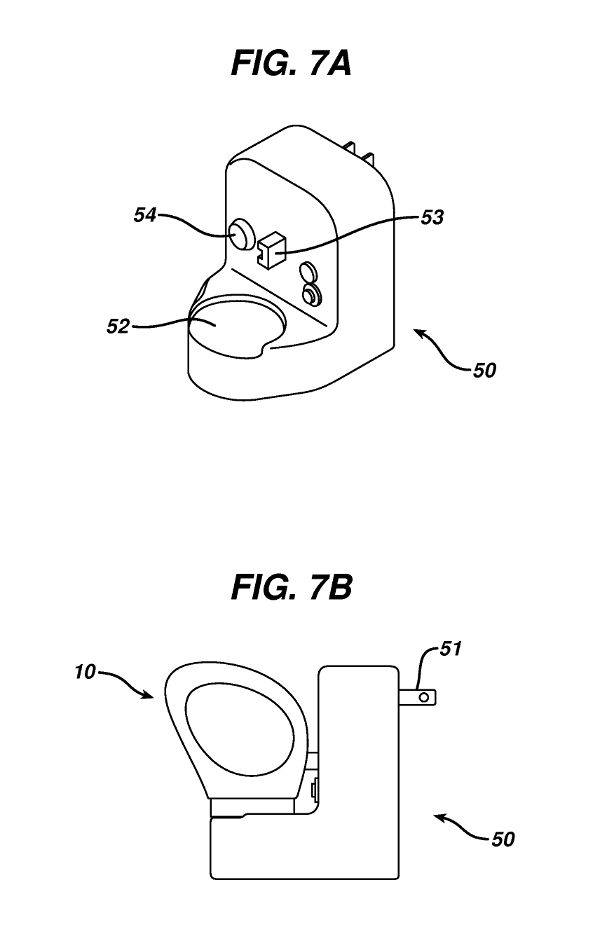

Referring now to FIG. 7A-7B, the skin care device 10 can be connected to a combination stand and energizer (or “energizer stand”) 50 that includes a plug 51 to electrically connect it to an external power source, such as a power grid (for example, through 110V household power). While plug 51 is shown as extending from a rear portion of the energizer stand 50, it will be recognized that this integrated plug may be replaced by a power cord and plug combination. The skin care device can be supported on a platform 52, and external electrical connector 53 is arranged and configured to engage the electrical contacts 23 (shown in FIG. 2) of the device 10 to energize the heat-generating element. The energizer stand 50 preferably includes an energizing circuit to control the energization of the heat-generating element. The energizing circuit may include a timer and may be initiated by activating an energizing circuit switch 54.

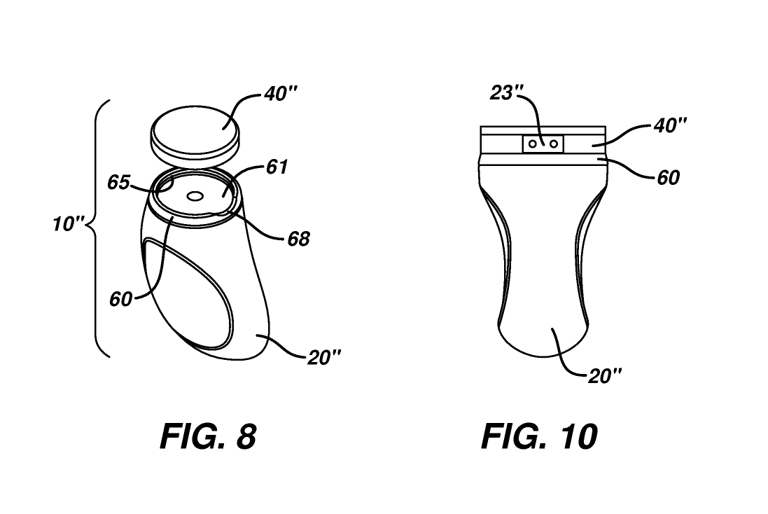

In an alternate embodiment shown in FIGS. 8-11, the housing 20″ further incorporates a mount 60 for the thermal body-care element 40″. The thermal body-care element 40″ is then operatively connected to the mount 60 on the housing 20″. In one embodiment, the thermal body-care element 40″ is releasably connected to the mount 60. The mount 60 secures the thermal body-care element 40″ to the housing 20″ without affecting the ability of the thermal body-care element 40″ to function as desired. For example, the mount 60 may rotate, oscillate, or provide other desired movement to the thermal body-care element 40″, or it may remain stationary on the housing 20″ to transfer desired vibrations from the motion-generating system 30. Suitable mounts will be known to those of ordinary skill in the art. A representative, non-limiting list of useful mounts include snap-fit mounts; threaded mounts; pinned, clipped, or ringed mounts (using a removable pin, peg, clip, or ring to immobilize the thermal body-care element in or to the mount), clamped mounts, bayonet mounts, hook-and-loop (VELCRO) mounts, and the like.

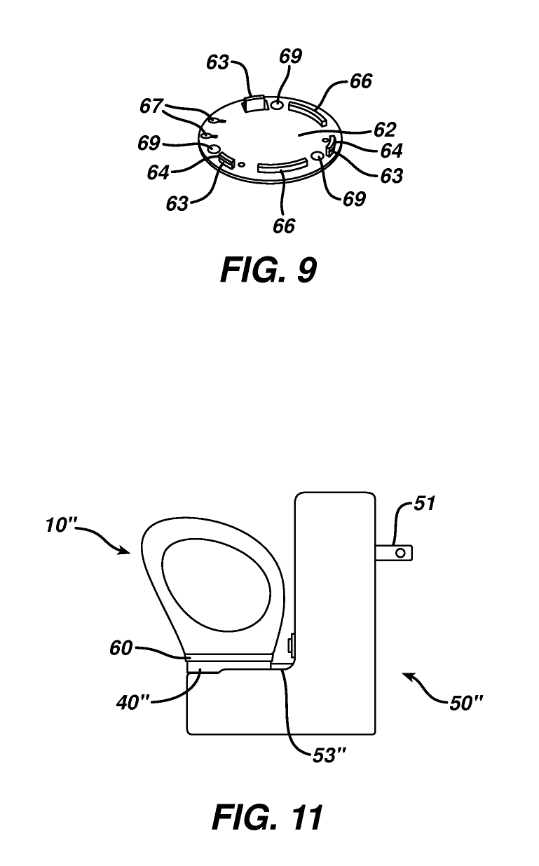

For example as shown in FIG. 8, the handheld device 10″ has a rotating mount 60 with a receptacle 61 into which coupling elements of the thermal body-care element 40″ fit. The thermal body-care element 40″ is substantially circular and has a diameter between about 20 mm and about 60 mm. The attachment surface 62 of the thermal body-care element 40″ designed to fit into the receptacle 61 of the mount 60 of this embodiment is shown in more detail in FIG. 9. The attachment surface 62 has a plurality of engagement arms 63 extending from the attachment surface 62 in a direction away from the thermal body-care element 40″, at least one of said engagement arms comprising a snap-fit projection 64 for engagement with recesses 65 of an associated receptacle 61 (shown in FIG. 8). At least one spacer leg 66 extends from the attachment surface 62 in a direction away from the thermal body-care element 40″ to support it when fitted into the associated receptacle 61. The attachment surface 62 also has at least one key 67 extending from the attachment surface 62 in a direction away from the thermal body-care element 40″ that is arranged and configured to fit into a notch 68 in the associated receptacle 61. The attachment surface 62 may also have one or more optional centering flange(s) 69 to improve the fit of the thermal body-care element 40″ in the receptacle 61. Alternatively, the functions of the spacer leg(s) and the centering flange(s) may be combined into one or more separate structures spaced about the attachment surface 62.

As shown in FIG. 10, the electrical contacts 23″ for selective coupling the heat-generating element to an external electrical power source are disposed on an outer surface of the thermal body-care element 40″. The skin care device 10″ of this embodiment can be connected to a modified energizer stand 50″ that includes a plug 51 to electrically connect it to an external power source as shown in FIG. 11. The skin care device can be supported on a platform, and external electrical connector 53″ is arranged and configured to engage the electrical contacts 23″ of the device 10 to energize the heat-generating element. Again, the energizer stand 50″ preferably includes an energizing circuit to control the energization of the heat-generating element. The energizing circuit may include a timer and may be initiated by activating an energizing circuit switch.

In greater detail, the heat source of the thermal body-care element is disposed within the thermal body-care element and is in thermal contact with the thermal energy storage medium. The heat source can be any heat source that receives energy from outside of the thermal body-care element and provides thermal energy. Preferred heat sources include without limitation, electrical heat sources, such as resistance heaters, positive temperature coefficient (“PTC”) heaters, cartridge heater (typically a resistance heater in a cartridge), and the like; electromagnetic heat sources, such as infrared heaters, and the like. More preferably, the heat source is an electrical heat source, and most preferably, the heat source is a Positive Temperature Coefficient heater (PTC). PTC heaters are known for their self-regulating features and can operate at a nearly constant temperature over a broad range of voltage and dissipation conditions by increasing their resistance as temperature increases. PTC's can permit the omission of thermostats in certain situations since PTC's increase resistance as the temperature increases. Alternatively, a fixed resistance heater would require a more sophisticated control system in order to maintain temperature and to overshooting its temperature set point. This design uses both a PTC heater and a simple bimetallic temperature switch for control. More specifically a bimetallic “creeps action” switch was used instead of a “snap action” in order to maintain the temperature within +/−3° C.

Additional safety can be achieved by incorporating a thermal switch. This provides the added safety in the event that both the bimetallic switch was to fail (not open) and the PTC heater somehow produced excess heat. The thermal switch would permanently open the circuit thus removing all energy.

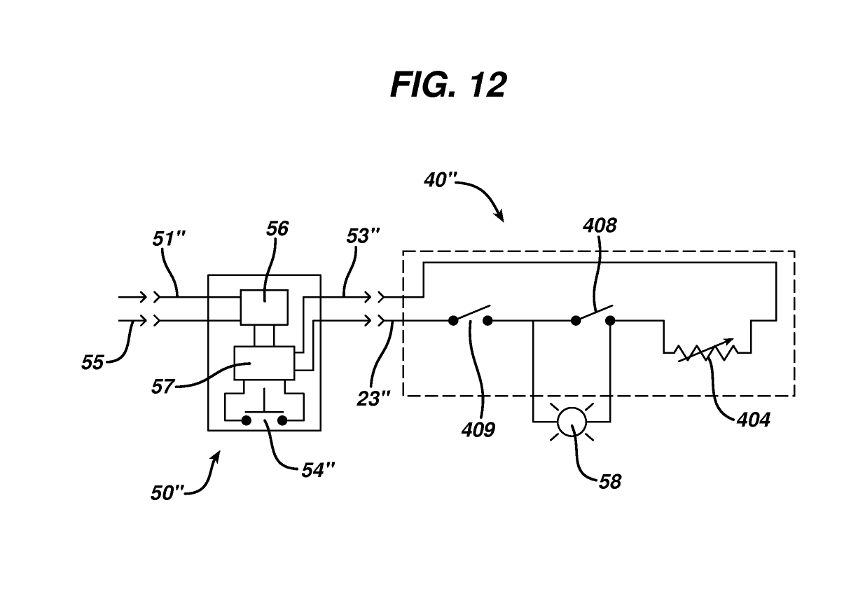

An example of a circuit for the energizer stand 50″ and thermal body-care element 40″ of FIGS. 8-11, is shown in FIG. 12. The energizer stand 50″ includes a plug 51″ for selective coupling to a power grid 55. The energizer stand 50″ also includes an energizing circuit including a transformer to convert alternating current to direct current and an appliance leakage circuit interrupter (shown schematically as 56. In addition, the circuit includes a timer 57 and a charging circuit switch 54″. The energizer stand 50″ is connected to the thermal body-care element 40″ through the external electrical connector 53″ and the thermal body-care element electrical contacts 23″. In this schematic, the thermal body-care element 40″ includes a safety cut-off switch 409, a thermal switch 408, and a PTC heater 404. An optional indicator light 58 is also shown.

To generate heat, the heat source requires energy to be delivered from outside of the thermal body-care element. One traditional method may include wire leads or other metallic connections from the heat source to the outside of the thermal body-care element. Other possibilities include electromagnetic induction. In one preferred embodiment, wire leads extend from the heat source to one or more couplers disposed on the thermal body-care element that can engage a connector of an outside power source. This power source may be a conduit through the housing to electrical contacts disposed thereon. These electrical contacts in turn are connected to an external power source, such as household electrical supply through a power plug.

Again, the thermal body-care element includes a container includes a base formed of a thermally insulating material and a thermally conductive cap. The container may also include additional thermal insulating surfaces, such as the external sleeve. A representative, non-limiting list of useful thermally insulating materials includes polymeric and other organic materials, ceramic materials, and the like. Preferred thermally insulating materials have low thermal and electrical conductivity, such as polymers and ceramics, and are dimensionally stable.

Thermally conductive materials useful for the cap and other thermally conductive elements of the container may be formed of any thermally conductive material. A representative, non-limiting list of useful thermally conductive materials includes thermally conductive polymeric materials, metals, and the like. Preferred thermally conductive materials include thermally conductive polymers that have low electrical conductivity.

The thermal energy storage medium is used to store the heat generated by the heat source while it is connected to the external power source and to release the heat to the body-care surface during use. Preferably, the thermal energy storage medium has a high heat storage density. There are three main physical ways for thermal energy storage: sensible heat, phase change reactions and thermo-chemical reactions. Sensible heat storage is based on the temperature change in the material and the unit storage capacity is equal to heat capacitance x temperature change. Sensible heat systems generally have a large thermal mass or require a large temperature range.

Phase change heat storage results when a material changes its phase while heating; the heat stored in the phase change is dissipated during cooling of the material as the phase change is reversed. The storage capacity of the phase change materials is equal to the phase change enthalpy at the phase change temperature+sensible heat stored over the whole temperature range of the storage.

Phase change systems generally have a much higher energy storage density than sensible heat storage systems.

Therefore, one preferred form of thermal energy storage medium is a phase change material. Preferred phase change materials have high latent heat of fusion per unit mass (increases energy storage density), high specific heat that provides additional sensible heat storage effect, high thermal conductivity, high density, and most significantly, a melting point at the desired operating temperature range. Additional considerations include safety (non-flammable, non-poisonous, non-explosive), non-corrosive, no chemical decomposition. Classes of phase change materials include organic and inorganic materials. Organic materials include, without limitation, waxes such as paraffins (CₙH₂ₙ₊₂) and fatty acids (CH₃—(CH₂)₂ₙ)—COON) such as lauric acid, stearic acid, pentaglycerine. These materials generally are chemically stable, have a high heat of fusion, are safe, are non-reactive, and are recyclable. However, they may suffer from relatively low thermal conductivity in their solid state, their volumetric latent heat storage capacity is relatively low; they may be flammable. In particular, paraffin waxes are available from many commercial sources. These waxes generally do not have a distinct melting point temperature. Rather, they have a melting point range. For example, one material available from Strahl & Pitsch, Inc. West Babylon, N.Y., USA has a melting point range (Melting Point Open Cap. Tube USP Class II) of 122-127° F. (50-53° C.). Other paraffin waxes may have a melting point range of up to 69-73° C.

One issue identified above in phase change material systems is significant change in volume in during the phase change and/or chemical reaction. Therefore, it may be desirable to employ a “pressure block” 411 (shown in FIG. 5C) or material that is capable of expanding or contracting to volume changes that can be created by an air pocket or the thermal energy storage medium. The pressure block 411 can be a separate item, attached to the cup or an integrated into the molding process of the thermal body care element. Another purpose of the pressure block is to help maintain the thermal energy storage medium in a specific location such as against the electric heater or on one side of the thermal body care element.

The thermal energy storage medium can be selected for its operating temperature range. Generally, a sensible heat system will be operable over a very broad temperature range—a range over which it does not change phase. A phase change system will have particularly usefulness when the operating temperature range spans the phase change temperature—especially a phase change temperature near and/or slightly above the desired heat application temperature

The amount of thermal energy storage medium used in the thermal body-care element will be determined by the available volume and the desired heat capacity of the thermal body-care element. In one preferred embodiment for use in a handheld device having a motion-generating system may include about 1 to about 5 g of a wax or wax mixture. More preferably, this embodiment may include about 1 to about 3 g of the wax or wax mixture.

In alternative embodiments, such as the hot stone shown in FIGS. 6A and 6B, described above, the amount of wax may be increased. For example, one size of hot stone may include about 2 to about 12 g wax or wax mixture, and more preferably, between about 3 and about 10 g of the wax or wax mixture.

Supplemental body-care component 46 may be a pad or other element that is capable of transferring heat from the heat-generating element 41 and that is placed in association with the body-care surface 42. The supplemental body-care component 46 may be mated to the body-care surface 42 and may include a securing surface for contacting a coupling structure 47 of the body-care surface 42 to temporarily secure the supplemental body-care component 46 to the body-care surface 42 during operation of the device. As discussed above, the securing surface may be engaged by the coupling structure.

Alternatively, the supplemental body-care component 46 may be held in place during operation of the device via any number of suitable mechanical or magnetic components (not shown), such as clamps, snaps, adhesive, and the like may be used to facilitate the attachment and detachment of the supplemental body-care component 46.

In one preferred embodiment, the supplemental body-care component 46 is or includes a porous material, such as a porous sheet. The pores may be capable of transporting liquid from within the supplemental body-care component 46 to a skin-contactable surface thereof. The sheet may be fibrous and/or film-based (e.g., may include fibrous and/or plastic film materials, such as one or more layers of these materials). The layers of material may be relatively rigid or relatively compliant and may serve one or more functions such as enhancement of friction by the skin-contactable surface on the skin, transport of sebum away from the skin, transport of various cleansers and/or benefit agents, as described below, towards the skin so that they may provide some benefit thereto, among other functions. Suitable fibrous materials that may be used include those based from organic polymers such as, for example, polyester, polyolefin, rayon, cellulose such as from wood pulp, bicomponent fibers, and other combinations thereof. The fibers are woven or non-woven and arranged in a network via, for example, a carding process, and bonded via, for example, an air-through bonding, chemical bonding, or an embossing process. The layer of fibrous material may include binders such as organic resins or other ingredients to manipulate the mechanical or fluid management properties thereof. The layer of fibrous material may have a basis weight that supports the layer to maintain its mechanical integrity for one or more uses of the supplemental body-care component 46. The basis weight may be, for example, between about 10 grams per square meter (gsm) and about 100 gsm, such as between about 40 gsm and about 60 gsm.

Alternatively, the supplemental body-care component 46 may have massaging protrusions, oriented fibers (such as a brush or dilour surface), and/or a coated surface. However, it may be desirable to have the massaging protrusions and/or coated surface be the body-care surface 42, itself.

In one embodiment of the invention, the supplemental body-care component 46 includes one or more cleansers and/or benefit agents. Various cleansers are known to those of ordinary skill in the art, and the chosen cleanser (if any) is not critical to the operation of the present invention. What is meant by an “benefit agent” is a compound (e.g., a synthetic compound or a compound isolated from a natural source) that has a cosmetic or therapeutic effect on the skin including, but not limited to, lightening agents, darkening agents such as self-tanning agents, anti-acne agents, shine control agents, anti-microbial agents, anti-inflammatory agents, antifungals, anti-parasite agents, external analgesics, sunscreens, photoprotectors, antioxidants, keratolytic and exfoliating agents, surfactants, moisturizers, nutrients, vitamins, energy enhancers, anti-perspiration agents, astringents, deodorants, hair growth inhibitors, anti hair-loss agents, hair growth promoters, hair removers, skin-firming agents, anti-callous agents, anti-aging agents such as anti-wrinkle agents, skin conditioning agents, allergy inhibitors, antiseptics, external analgesics, antipruritics, antihistamines, antiinfectives, anticholinergics, vasoconstrictors, vasodilators, wound-healing promoters, peptides, polypeptides, proteins, deodorants, anti-perspirants, film-forming polymers, counterirritants, enzymes, enzyme inhibitors, poison ivy treatment agents, poison oak treatment agent, burn treatment agents; anti-diaper rash treatment agents; prickly heat agents; botanical extracts including herbal extracts; flavenoids; sensates; anti-oxidants, keratolytics; sunscreens; and anti-edema agents; and combinations thereof.

What is meant by a “botanical extract” is a blend of two or more compounds isolated from a plant. Examples of botanical extracts include, but are not limited to legumes such as Soy, Aloe Vera, Feverfew, Hedychium, Rhubarb, Portulaca, Cedar Tree, Cinnamon, Witch Hazel, Dandelion, Chinese Angelica, Turmeric, Ginger, Burnet, Houttuynia, Coix Seed, and Thyme.

In one embodiment of the invention, the benefit agent is designed for application on the forehead region and includes, but is not limited to: oil-control agents such as titanium dioxides, alcohols, botanical extracts, and talc; pore refining agents such as alpha-hydroxy acids, beta-hydroxy acids, and enzymes; anti-acne agents such as benzoyl peroxide, salicylic acid, trichlorcarban, triclosan, azelaic acid, clindamycin, adapalene, erythromycin, sodium sulfacetamide, retinoic acid, and sulfur; oil-absorbing agents such as titanium dioxides and clays; shine control agents such as silicones, alcohols, talc, and clays; dark spot reduction agents such as vitamin C, hydroquinone, botanical extracts, alpha-hydroxy acids, beta-hydroxy acids, and retinoids; and/or wrinkle/fine-line reduction agents such as retinoids, alpha-hydroxy acids, and enzymes.

In another embodiment of the invention, the benefit agent is designed for application around the mouth and includes, but is not limited to: hydration/moisturization agents such a glycerin, silicone, glycols, botanical extracts, and esters; pore-refining agents; anti-acne agents; vasodilators such as niacinamide and horsechesnut extract; vasoconstrictors such as caffeine and botanical extracts; skin-lifting agents such as (e.g., copper containing peptides, dimethyaminoethanol, and polymers); skin-firming polymers; wrinkle/fine-line reduction agents; depigmenting/skin lightening agents such as vitamin C, hydroquinone, botanical extracts, alpha-hydroxy acids, beta-hydroxy acids, retinoids, arbutin, and kojic acid; and depilatory/hair reducing agents such as soy extracts, n-acetyl-cysteine, and isoflavones.

In order to use the system of the present invention, a user may connect the energizer stand 50 to household current through plug 51. In a preferred embodiment, the user would power the system by pressing the energizing circuit switch 54 to enable electrical current to flow into the thermal body-care element 40. When the thermal body-care element 40 is fully energized to provide the desired heat, a signal may alert the user to remove the handheld skincare device 10 from the energizer stand 50.

If desired, the optional, supplemental body-care component 46 may be applied to the body-care surface 42 of the thermal body-care element 40. In addition, the supplemental body-care component 46 may be moistened or wetted by running under tap water. The supplemental body-care component may be applied any time before use. For example, the supplemental body-care component 46 may be applied before energizing the thermal body-care element 40, or it may be applied after the thermal body-care element 40 has been fully energized and heated, immediately prior to application to the user's skin.

The motion-generating system 30 can be activated by the user, and the skin care device 10 can be moved about in contact with the user's skin for the desired effect.

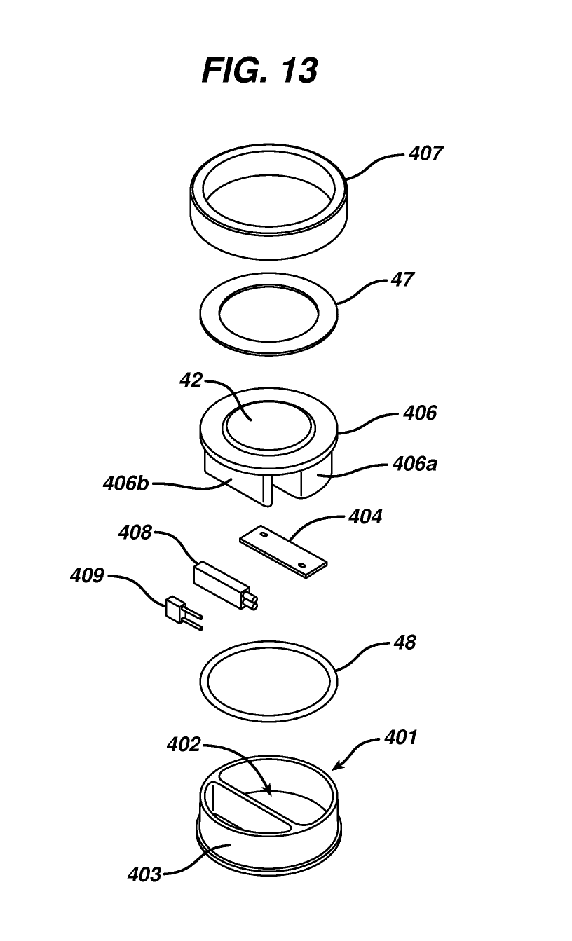

One way to make the thermal body-care element 40 of FIG. 5 is shown in FIG. 13. A thermally-insulating, cylindrical cup 401 having two chambers is formed of plastic and has external threads. A PTC heater 404 is secured to the plastic base of the first chamber 402.

A thermally conductive cap 406 has walls 406a arranged and configured to line the first chamber of the cup 401 when assembled. A flange 406b extends from the top surface of cap 406 into the second chamber 403 of the cup 401. A thermal switch 408 and safety cut-off switch 409 are secured to the flange to enable them to sense and control the temperature in the first chamber 402 via the thermal conductivity of the cap 406.

A gasket 48, such as an o-ring, is disposed on the top of the cup 401, and the cap 406 with the thermal switch and safety cut-off switch mounted on the flange is placed on the cup 401. The heater, thermal switch and safety cut-off switch are electrically interconnected, e,g, via insulated wires that pass through the wall 401a dividing the two chambers, in an electrical circuit. The electrical circuit also has electrical contacts 23″ 410 to engage the external power source.

An optional coupling structure 47, such as plastic hooks may be adhered to a portion of the body-care surface 42 of the cap 406, and a threaded external sleeve 407 of the thermal body-care element 40 is screwed onto the external threads of the cup 401 to complete the container 43. A thermal energy storage medium 405, such as wax, can be injected through a port into the enclosed first chamber, and the port can be sealed to complete the assembly.

The foregoing is only one way to make the thermal body-care element of the present invention. Persons of ordinary skill will recognize various alternatives of assembling the inventive thermal body-care element.

### EXAMPLES

### Example 1

The following is an example of the determination of the mass of paraffin wax needed to provide a desired amount of heat for a predetermined period of time. For this example, the values are approximate and rounded to no more than 2 significant figures. In this example, we show a process to determine the system necessary to heat a wetted pad (2 grams of water held by a small nonwoven pad) from 20° C. to 40° C. and providing that heat for 120 seconds. Based upon the mass of water, temperature range, duration of heat and an assumed 50% efficiency in transferring heat to the wetted pad, we determined that 450 Joules (“J”) of energy was required.

In this example, we used a paraffin wax having a specific heat of about 2000 J/(kg-° C.) in the liquid phase, about 1000 J/(kg-° C.) in the solid phase, and a heat of fusion of about 210000 J/kg, and a melting point range of about 53-57° C. We assumed an initial liquid phase temperature of about 60° C., and a final solid phase temperature of about 40° C. when used to warm the wetted pad. In order to provide the 450 J to the pad, we determined that about 0.002 kg (2 g) of the wax was required.

In order to store 450 J of thermal energy in 2 g of the paraffin wax, we determined the amount of electrical energy necessary to heat 2 g of the paraffin wax from an initial solid phase temperature of 20° C. to a final liquid phase temperature of about 55-60° C. In order to minimize the electrical energy required, we chose to maximize thermal transfer efficiencies by using an internal electric heater surrounded by the wax. Based upon assumed operating efficiencies and assumed thermal transfer efficiencies, we determined the electrical energy required to store 450 J of thermal energy in 2 g of the wax to be about 1000 J. Finally, we set a target time to heat the wax of 90 seconds. Therefore, the electrical power necessary to provide and store the heat energy is 1050 J/90 s, or about 12 Watts (“W”).

In order to determine whether the energy source to provide the necessary electrical power [Power (W)=Voltage (V)*Current (I)] could have been provided in the cordless, handheld skin-care device, we made the following assumptions and calculations. Assuming a 1.5 V single cell battery is used, the current necessary to generate 12 W would be:

12 W=1.5 V*I,

or

I=12 W/1.5 V,

or

I=8 Amps

As AA batteries operate in the range of milliamps (“mA”), a large number of AA batteries would be required. Larger capacity batteries are too large and heavy and/or too expensive. Therefore, an external power source, such as at 120 VAC, was selected. At 120 VAC, the required current would be orders of magnitude lower:

I=12 W/120 V,

or

I=0.1 Amps (100 mA)

Thus, the external power source permits the use of low currents and consequently smaller components.

### Example 2

The following is an example of the determination of the mass of paraffin wax needed for an embodiment of a hot stone for placement on skin for 10 to 30 minutes.

Again, we used a paraffin wax having a specific heat of about 2000 J/(kg-° C.) in the liquid phase, about 1000 J/(kg-° C.) in the solid phase, and a heat of fusion of about 210000 J/kg, and a melting point range of about 53-57° C. We assumed an initial liquid phase temperature of about 60° C., and a final solid phase temperature of about 40° C. when used to warm the wetted pad. We also assumed that the heat energy was transferred to the skin at 1 J/sec or 1 W. Thus, for a duration of 10 minutes, 600 J of heat energy was required. For a duration of 30 minutes, 1800 J was required. This results in a mass of wax of about 2.5 g (10 minute application) to about 8 g (30 minute application). Of course, the electrical power required to heat and store the heat in the wax (and larger container) would be increased from Example 1.

The specification and embodiments above are presented to aid in the complete and non-limiting understanding of the invention disclosed herein. Since many variations and embodiments of the invention can be made without departing from its spirit and scope, the invention resides in the claims hereinafter appended.

What is claimed is:

1. A thermal body-care element comprising:
a) a container having at least one thermally conductive surface, the container enclosing an electric heater in thermal contact with a thermal energy storage medium comprising a phase change storage medium; and
b) an electrical connection for selective coupling to an external electrical power source;
wherein the external electrical power source is external to the thermal body-care element.

2. The thermal body-care element of claim 1, wherein the electric heater is selected from the group consisting of an electrical resistance heater, a positive temperature coefficient heater, a cartridge heater, and combinations thereof.

3. The thermal body-care element of claim 2, wherein the electric heater is a positive temperature coefficient heater.

4. The thermal body-care element of claim 1, further comprising temperature control elements.

5. The thermal body-care element of claim 4, wherein the temperature control elements comprise a creep action switch.

6. The thermal body-care element of claim 1, wherein the thermal energy storage medium further comprises a sensible heat storage medium.

7. The thermal body-care element of claim 1, wherein the phase change storage medium comprises a wax or a wax mixture.

8. The thermal body-care element of claim 1, wherein the thermal energy storage medium further comprises a pressure block.

9. A system comprising the thermal body-care element of claim 1 and an energizing stand.

10. The system of claim 9, wherein the energizing stand engages the electrical connector of the thermal body-care element to energize the thermal body-care element, and the energizing stand is selectively coupleable to a power grid.

11. The thermal body-care element of claim 1, wherein the thermally conductive surface is arranged and configured to hold a supplemental body-care component.

12. The thermal body-care element of claim 11, wherein the supplemental body-care component comprises a porous material.

13. The thermal body-care element of claim 11, wherein the supplemental body-care component further comprises at least one cleanser, at least one skin benefit agent, or combinations thereof.

14. The thermal body-care element of claim 1 which is arranged and configured for releasable attachment to a handheld device comprising a motion-generating system.

15. The thermal body-care element of claim 14, comprising about 1 to about 5 grams of the phase change storage medium.

16. The thermal body-care element of claim 1 which is arranged and configured for placement on a mammalian body in a hot stone massage process.

17. The thermal body-care element of claim 16, comprising about 2 to about 12 grams of the phase change storage medium.

18. A method of applying heat to a skin surface of a human or animal comprising the steps of:
a) electrically connecting an electric heater to an external electrical power source to heat a thermal energy storage medium in thermal contact therewith, the electric heater and thermal energy storage medium enclosed in a container arranged and configured for gripping by a human hand;
b) disconnecting the electric heater from the external electrical power source; and
c) applying the body-care surface to the skin surface while the electric heater is disconnected from the external electrical power source.

19. The method of claim 18, further comprising the step of coupling a supplemental body-care component to the body-care surface.

20. The method of claim 18, wherein the electric heater is a positive temperature coefficient heater.

21. The method of claim 18, wherein the thermal energy storage medium comprises a sensible heat storage medium, and the step of heating the thermal energy storage medium comprises raising the temperature of the sensible heat storage medium.

22. The method of claim 18, wherein the thermal energy storage medium comprises a phase change storage medium, and the step of heating the thermal energy storage medium comprises changing the phase of the phase change storage medium.

23. The method of claim 22, wherein the phase change storage medium is selected a wax or a wax mixture, and the step of heating the thermal energy storage medium comprises melting the wax the phase of the phase change storage medium.
